(19) United States
(12) Patent Application Publication
(10) Pub. No.: US 2025/0011854 A1
(43) Pub. Date: Jan. 9, 2025

(54) Methods of producing DNA templates for mRNA production

(71) Applicant: SMOBIO Technology, Inc., Hsinchu City (TW)

(72) Inventors: CHEN-SHENG WU, Hsinchu City (TW); KUAN-CHUN CHEN, Hsinchu City (TW); CHUN-HSIEN KUO, Hsinchu City (TW); RICHARD K. LEE, Hsinchu City (TW)

(73) Assignee: SMOBIO Technology, Inc., Hsinchu City, TW (TW)

(21) Appl. No.: 18/761,337

(22) Filed: Jul. 2, 2024

Related U.S. Application Data
(60) Provisional application No. 63/511,692, filed on Jul. 3, 2023.

(51) Int. Cl. C12Q 1/6855; C12Q 1/44; C12Q 1/686

(52) U.S. Cl. CPC C12Q 1/6855; C12Q 1/44; C12Q 1/686; G01N 2333/922

(57) ABSTRACT

The invention provides a method for producing a DNA template for mRNA containing a polyadenylate tail. This method is applicable to in vitro transcription reactions, generating mRNA with a specific polyadenylate tail length. Additionally, the invention further provides a method for extending the polyadenylate tail sequence using terminal deoxynucleotidyl transferase and combinations of restriction enzymes. This ensures that in in vitro transcription reactions, the mRNA can precisely terminate at the polyadenylate tail, thereby improving the accuracy and efficiency of mRNA synthesis.

This application contains a Sequence Listing in a computer readable form, the file name is sequence listing, created on Jul. 1, 2024, the size is 31,000 bytes, which is incorporated herein by reference.

### FIELD OF THE INVENTION

The present invention relates to the field of mRNA molecular technology, particularly to a production method for DNA templates used for producing mRNA containing polyadenylate tails.

### BACKGROUND OF THE INVENTION

The manufacturing process of mRNA therapeutic drugs for clinical use must produce high-purity and high-potency mRNA with consistency, reproducibility, and compliance with current Good Manufacturing Practices. The mRNA must be as consistent as possible, including having a uniform 5′ cap structure (5′-cap), the correct sequence, and the correct polyadenylate (poly(A)) tail length while minimizing the generation of related impurities.

During the RNA processing, to increase stability, a long chain of adenine nucleotides (poly(A) tail) may be added to polynucleotides, such as mRNA molecules. After transcription, the 3′ end of the transcript may be cleaved, releasing a 3′-hydroxyl group, and then poly(A) polymerase adds a chain of adenine nucleotides to the RNA. This process, known as polyadenylation, typically results in a poly(A) tail length of 100 to 250 residues. Poly(A)-binding protein (PABP) binds to the poly(A) tail and interacts with the eukaryotic initiation factor 4F (eIF4F) protein complex bound to the cap, forming part of the translation initiation complex. The poly(A) tail works in conjunction with the 5′-cap and other various determinants to regulate the translation efficiency and stability of mRNA.

In order to produce mRNA containing a polyadenylate tail, several methods utilizing in vitro transcription have been adopted. One method involves transcribing a DNA template without a polyadenylate tail and then using poly(A) polymerase to add a 3′ polyadenylate tail to the transcribed mRNA, typically exceeding 100 bases. However, the drawback of this method is the difficulty in controlling the tail length, resulting in mRNA of varying lengths, thereby increasing the complexity of subsequent purification and making it challenging to maintain a high-quality mRNA product.

To overcome this drawback, another method involves adding an encoded poly(A) tail to the DNA template vector, so that the mRNA transcript immediately has a poly(A) tail. However, for each mRNA, it is necessary to design a DNA template with the appropriate poly(A) tail length. Therefore, developing a simple and flexible method for DNA templates is an important technical means for producing mRNA.

The method of adding a poly(A) tail to the DNA template typically involves constructing it on a plasmid. During the construction process, the length of the poly(A) structure can affect the stability of the plasmid in E. coli, often making it difficult to obtain longer poly(A) sequences. Therefore, this invention proposes a strategy to link the target sequence with the poly(A) tail using polymerase chain reaction (PCR) or ligation reactions to accelerate or enhance the construction of DNA templates with poly(A) tails, thereby reducing subsequent production steps and costs.

### DETAILED DESCRIPTION OF THE INVENTION

The present invention provides a method for producing a DNA template, wherein the DNA template is used for producing mRNA containing a polyadenylate tail, the DNA template comprising: providing a first DNA template, wherein the first DNA template comprises, in sequence from the 5′ end to the 3′ end, a forward primer sequence, a promoter, a target DNA, wherein the target DNA further comprises a 5′ untranslated region and a 3′ untranslated region, a polyadenylate, a first restriction enzyme cutting site, and a specific sequence, wherein the specific sequence is a reverse primer complementary sequence; using the first DNA template as a template, adding a forward primer and a reverse primer to perform PCR amplification of the first DNA template; and performing a cleavage on amplified the first DNA template using the first restriction enzyme, wherein the cleavage removes the specific sequence of the first DNA template to obtain the DNA template. The first DNA template can also be designed in another form as shown in FIG. 1B, with the sequence having a second restriction enzyme cutting site between the 3′ untranslated region and the polyadenylate, this restriction enzyme cutting site is referred to as the second restriction enzyme cutting site.

According to the DNA template production method provided by the present invention, the DNA template can be used as a DNA template for in vitro transcription (IVT) to produce mRNA with a specific length of polyadenylate tail.

In one embodiment, the promoter is a T7, T3, or Sp6 bacteriophage RNA polymerase promoter.

In one embodiment, the forward primer sequence may be partially or entirely identical to a segment of the promoter sequence.

According to the DNA template production method provided by the present invention, the length of the polyadenylate is greater than 100 nucleotides; the length of the polyadenylate is greater than 200 nucleotides; the length of the polyadenylate is greater than 300, 400, 500, 600, and 1000 nucleotides; furthermore, the length of the polyadenylate can be adjusted proportionally based on different mRNA lengths.

According to the DNA template production method provided by the present invention, the method for producing the first DNA template is obtained through ligation reaction or overlap PCR using a Second DNA template and a Third DNA template; wherein the Second DNA template comprises, in sequence from the 5′ end to the 3′ end, a forward primer sequence, a promoter, a target DNA, wherein the target DNA further comprises a 5′ untranslated region and a 3′ untranslated region; wherein the Third DNA template comprises, in sequence from the 5′ end to the 3′ end, a 3′ untranslated region sequence that is “identical overlapping” or “partially continuous” with the Second DNA template, a polyadenylate, a first restriction enzyme cutting site, and a specific sequence, wherein the specific sequence is a reverse primer complementary sequence; wherein “identical overlapping” refers to the 3′ untranslated region sequences of the Second DNA templates and Third DNA templates having identical parts for the execution of overlap PCR, and “partially continuous” refers to the 3′ untranslated region sequences of the Second and Third DNA templates being non-redundant continuous sequences, which can be ligated using blunt-end ligation.

According to the DNA template production method provided by the present invention, the ligation reaction for joining the Second and Third DNA templates can be Gibson Assembly, overlap PCR, blunted end ligation, or sticky end ligation. The ligation reaction includes blunt-end direct ligation to form a complete 3′ untranslated region, or sticky end ligation after cleaving at restriction enzyme sites either inherent in or specifically designed between the 3′ untranslated region sequence and the polyadenylate (FIG. 2B).

According to the DNA template production method provided by the present invention, the invention provides a method for increasing the length of the polyadenylate in the Third DNA template. The method comprises: acting on the tail end of the Third DNA template sequence with terminal deoxynucleotidyl transferase (TdT) to add polyadenylate (poly(A)) or polythymidylate (poly(T)) sequences, and then amplifying the sequences using PCR, thereby obtaining polyadenylate sequences of different lengths. This method, depending on whether the R2 restriction enzyme cutting site is designed, can be divided into two forms, A and B, as shown in FIG. 3A and FIG. 3B.

According to one of the aforementioned embodiments, another embodiment is provided for increasing the length of the polyadenylate in the Third DNA template. The method comprises: (a) performing a first restriction enzyme digestion reaction: providing a first Third DNA template, adding a first restriction enzyme to perform a cleavage, generating the first Third DNA template with the first restriction enzyme cleavage, wherein the first Third DNA template with the first restriction enzyme cleavage exposes a sequence cleaved by the first restriction enzyme; (b) performing a second restriction enzyme digestion reaction: providing a second Third DNA template, adding a second restriction enzyme to perform a cleavage, generating the second Third DNA template with the second restriction enzyme cleavage, wherein the second Third DNA template with the second restriction enzyme cleavage exposes a sequence cleaved by the second restriction enzyme, wherein the sequences cleaved by the first and the second restriction enzymes are complementary sequences; and (c) performing a ligation reaction: performing a ligation reaction between the first Third DNA template with the first restriction enzyme cleavage and the second Third DNA template with the second restriction enzyme cleavage, obtaining the Third DNA template with increased polyadenylate length.

According to the method of the aforementioned embodiment, the combination of the first restriction enzyme and the second restriction enzyme in the Third DNA template is such that the sequences exposed at the ends after cutting by the two restriction enzymes are complementary. After ligation through the complementary sequences, the resulting combination cannot be cut by either restriction enzyme again.

In one embodiment, the combination of the first restriction enzyme and the second restriction enzyme in the template containing the polyadenylate fragment can be a combination of BamHI and BagII; a combination of any two of Spel, AvrII, Nhel, and XbaI; a combination of MluI and BssHII; a combination of any two of AgeI, XmaI, NgoMVI, and BspEI; a combination of any two of PciI, NcoI, and BspHI; a combination of MfeI and EcoRI; and a combination of NsiI and PstI.

According to one of the aforementioned embodiments, another embodiment is provided for increasing the length of the polyadenylate in the Third DNA template. The method comprises: (a) providing a first Third DNA template with only one restriction enzyme cutting site, the first restriction enzyme cutting site being reverse BspQI (BQ′) sequence, denoted as BQ′ for its reverse characteristic; providing a second Third DNA template with two restriction enzyme cutting sites, the second restriction enzyme cutting site being a BspQI (BQ) sequence, and the first restriction enzyme cutting site being a reverse Ear1 (Ea′) sequence; (b) cleaving the first Third DNA template with BspQI to generate the first Third DNA template with BspQI cleavage, wherein the first Third DNA template with the BspQI cleavage exposes a sequence cleaved by BspQI, the overhang sequence being TTT; (c) cleaving the second Third DNA template with BspQI to generate the second Third DNA template with a BspQI cleavage, wherein the second Third DNA template with the BspQI cleavage exposes a sequence cleaved by BspQI, the overhang sequence being AAA; (d) ligating the first Third DNA template with the BspQI cleavage and the second Third DNA template with the BspQI cleavage to obtain a third Third DNA template; (e) cutting the third Third DNA template with Earl to generate the third Third DNA template with an Earl cleavage, wherein the third Third DNA template with the BspQI cleavage exposes a sequence cleaved by BspQI, the overhang sequence being TTT; and (f) ligating the third Third DNA template with the Earl cleavage and the second Third DNA template with the BspQI cleavage to obtain the Third DNA template with an extended polyadenylate. By repeating the above steps, the polyadenylate length can be further extended. This embodiment can also introduce a R2 restriction enzyme cutting site between the 3′ untranslated region sequence and the polyadenylate, as shown in FIG. 5B, to provide one of the methods for combining the Second and Third DNA templates.

The method described in the present invention involves an embodiment of a template containing a polyadenylate fragment. This template has a combination feature, including a first restriction enzyme and a second restriction enzyme, where their cutting positions are located outside the recognition sequences, rather than within the recognition sequences. Therefore, this combination feature is suitable for the splicing operation of polyadenylate (poly(A)).

According to the method of the aforementioned embodiment, the combination of the first and the second restriction enzymes BspQI and SapI are isoschizomers, having the same recognition sequence, and one more base pair compared to the recognition sequence of EarI. Therefore, EarI can cleave the BspQI (SapI) sequence, but BspQI (SapI) can only cleave its own recognized sequence.

According to the method of the aforementioned embodiment, there are EarI and BspQI cutting sites on both sides of the sequence containing the poly(A) fragment. Therefore, after ligation, the other side of the poly(A) sequence still retains the EarI sequence. This EarI cutting site can be used to extend the poly(A) sequence, or it can be used to remove the sequence after the poly(A) sequence before performing the IVT reaction, thus serving as the role of R1 in the aforementioned invention.

According to one of the aforementioned embodiments, another embodiment is provided for increasing the length of the polyadenylate in the Third DNA template. The method includes: (a) providing a first double-stranded oligonucleotide pairing sequence, comprising a portion of the 3′ untranslated region sequence and a fixed length of A/T pairing, with a protruding TTT, this double-stranded oligonucleotide pair can be formed by combining two artificially synthesized longer polynucleotides; (b) providing a second double-stranded oligonucleotide pairing sequence, comprising a fixed length of A/T pairing, a BspQI restriction enzyme cutting site, and a specific sequence (S-seq), with a protruding AAA at the A/T pairing position, this short nucleotide fragment can be formed by combining synthesized longer primers; (c) phosphorylating the 5′ ends of the primers with protruding TTT and AAA in the two double-stranded oligonucleotide pairs, respectively; (d) mixing the two double-stranded oligonucleotide pairs, and due to the complementary nature of AAA and TTT, ligating them to obtain a polyadenylate of double the length, as shown in FIG. 6A; (e) further amplifying this extended polyadenylate fragment using primers on both sides and cleaving with BspQI, resulting in a product with exposed TTT, and further pairing and ligating with the second double-stranded oligonucleotide pair's protruding AAA to extend further, repeating the above steps to lengthen the polyadenylate. This method can also be designed with a second restriction enzyme cutting site on the first double-stranded oligonucleotide pair, as shown in FIG. 6B, and the resulting Third DNA template can also be combined with the Second DNA template through the aforementioned second restriction enzyme cutting site.

The method described in the present invention involves an embodiment of a template containing a polyadenylate fragment. This template has a combination feature, including a first restriction enzyme with its cutting position located outside the recognition sequence, rather than within the recognition sequence. Therefore, this combination feature is suitable for the splicing operation of polyadenylate (poly(A)), and the initial short nucleotide fragment can be sourced from the ligation of longer primers.

According to the method of the abovementioned, the BspQI on the 3′ side of the poly(A) fragment can not only be used to extend the length of the poly(A) but also to remove the sequence following the poly(A) before performing the IVT reaction, thus serving as the role of R1 in the abovementioned invention.

According to the DNA template production method provided by the present invention, to ensure that the in vitro transcription reaction can stop at the position of the poly(A), restriction enzyme cutting sites such as HindIII, EcoRI, NsiI, SapI, XbaI, BspQI, or EarI can be designed at the end of the poly(A) tail. In this way, during the transcription reaction, it can precisely stop at the tail position of the poly(A), thereby avoiding or reducing the occurrence of other sequences being present at the tail of the poly(A) sequence.

The DNA template obtained in the present invention can be used as a DNA template for in vitro transcription of mRNA.

### BRIEF DESCRIPTION OF THE DRAWINGS

To make the above and other objectives, features, advantages, and embodiments of the present invention more apparent and understandable, the accompanying drawings are described as follows:

FIG. 1A and FIG. 1B illustrate the disclosed method for producing DNA templates according to the present invention.

FIG. 2A and FIG. 2B are embodiments of producing DNA templates.

FIG. 3A and FIG. 3B illustrate Method A for producing the Third DNA template. FIG. 3C-1 shows the electrophoresis diagram of the PCR amplification product in Example 3, Method A. FIG. 3C-2 presents the sequencing results after cloning into a vector in Example 3, Method A (SEQ ID NO: 17). FIG. 3C-3 depicts the expected sequence diagram in Example 3, Method A (SEQ ID NO: 18).

FIG. 4A illustrates Method B for extending the length of poly(A) through restriction enzyme cutting and ligation. FIG. 4B shows the Third DNA template with two restriction enzyme cutting sites in Method B. FIG. 4C presents the electrophoresis results after restriction enzyme cutting in Method B. FIG. 4D shows the sequencing results after extending the poly(A) in Method B. 60A #6 (1× poly A copy) is SEQ ID NO: 19. 120A #5 (2× polyA copy) is SEQ ID NO: 20. 180A #5 (3× polyA copy) is SEQ ID NO: 21. 240A #18 (4× polyA copy) is SEQ ID NO: 22.

FIG. 5A illustrates Method C for producing the Third DNA template. FIG. 5B shows another Method C for producing the Third DNA template. FIG. 5C-1 presents the electrophoresis results of changing the BglII cutting site to a reverse BspQI restriction enzyme cutting site in Method C. FIG. 5C-2 shows the electrophoresis results of changing the BamHI and BglII cutting sites to BspQI and reverse EarI restriction enzyme cutting sites, respectively, in Method C. FIG. 5D displays the sequencing results of different polyA lengths in Method C. 4A #70 is SEQ ID NO: 23. 103A #104 is SEQ ID NO: 24. 152A #124 is SEQ ID NO: 25.

FIG. 6A illustrates Method D for producing the Third DNA template. FIG. 6B shows another Method D for producing the Third DNA template. FIG. 6C presents the structure of the double-stranded oligonucleotide pair sequences in Method D. The final synthesized sequences include 6C-1 (SEQ ID NO: 26), 6C-2 (SEQ ID NO: 27), 6C-3 (SEQ ID NO: 28), and 6C-4 (SEQ ID NO: 29).

FIG. 7A shows the DNA fragment sequence containing the T7 promoter, 5′ UTR, green fluorescent protein open reading frame (GFP ORF), and 3′ UTR. GFP sequence (SEQ ID NO: 30). FIG. 7B presents the electrophoresis results of overlap PCR for combining the T7 promoter, 5′ UTR, GFP gene, and different polyA lengths (Method B). FIG. 7C shows the electrophoresis results after combining the T7 promoter, 5′ UTR, GFP gene, and different poly A lengths (Method C).

FIG. 8 shows the electrophoresis results of RNA after the capping reaction in the cell transfection experiment.

FIG. 9 shows the fluorescence microscopy observation results after cell transfection.

### Example

The practical application scope of the present invention can be further understood through the following detailed embodiments. These embodiments are merely preferred embodiments of the present invention and do not limit the scope of the present invention.

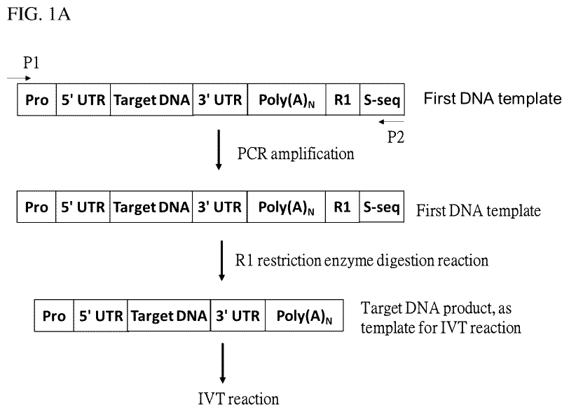

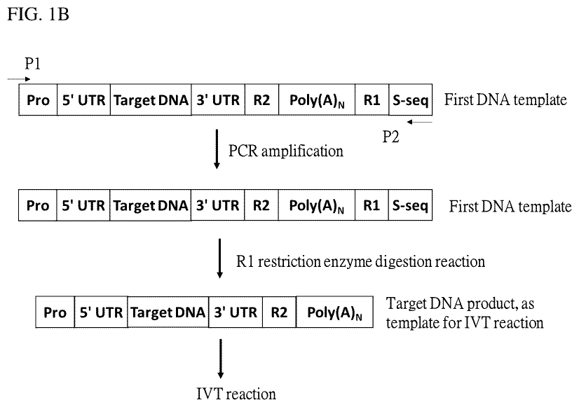

### Example 1: Method for Producing DNA Templates for In Vitro Transcription (IVT), as Shown in FIG. 1

Step 1: This DNA template can be amplified using primers. The amplification range and region of the primers cover the promoter (Pro) and the target DNA linked to the promoter, where the target DNA further includes a 5′ untranslated region (5′UTR) and a 3′ untranslated region (3′UTR), a polyadenylate (poly(A)_{N}), a first restriction enzyme cutting site (R1), and a specific sequence (S-seq) at the downstream end for PCR reaction. The entire DNA segment is amplified using the primer pair. The amplified DNA is then cut with a restriction enzyme to remove the sequence following the poly(A), resulting in a DNA template for the IVT transcription reaction. The difference between FIG. 1A and FIG. 1B is that the DNA template in FIG. 1B has a second restriction enzyme cutting site, due to different DNA construction methods, resulting in different final DNA templates.

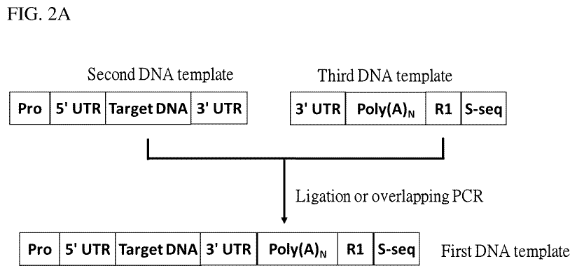

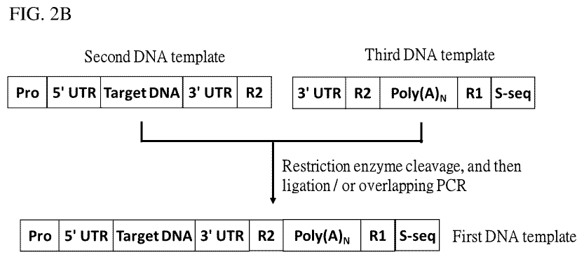

### Example 2: Method for Assembling and Generating DNA Templates, as Shown in FIG. 2A and FIG. 2B

Step 1: Provide a Second DNA template, wherein the Second DNA template comprises a promoter (Pro), and the promoter sequence includes a forward primer sequence, and the target DNA linked to the promoter, wherein the target DNA further comprises a 5′ untranslated region (5′UTR) and a 3′ untranslated region (3′UTR). Provide a Third DNA template, wherein the Third DNA template comprises the same 3′ untranslated region sequence (3′UTR) as the Second DNA template, a polyadenylate (poly(A)_{N}), a first restriction enzyme cutting site (R1), and a specific sequence (S-seq) at the downstream for PCR reaction.

Step 2: Obtain the First DNA template through ligation or overlapping PCR using the identical 3′ untranslated region on the Second DNA template and the Third DNA template. The difference between FIG. 2A and FIG. 2B is that FIG. 2B involves using restriction enzymes to cut the Second and Third DNA templates before ligation. This restriction enzyme can be designed between the 3′ untranslated region (3′UTR) and the polyadenylate (poly(A)_{N}), referred to as R2.

### Example 3: Method for Generating the Third DNA Template

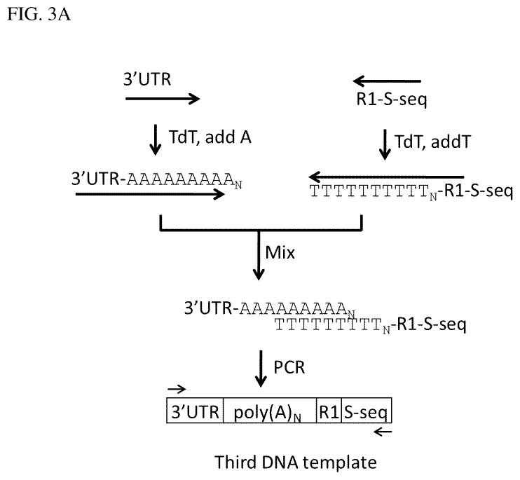

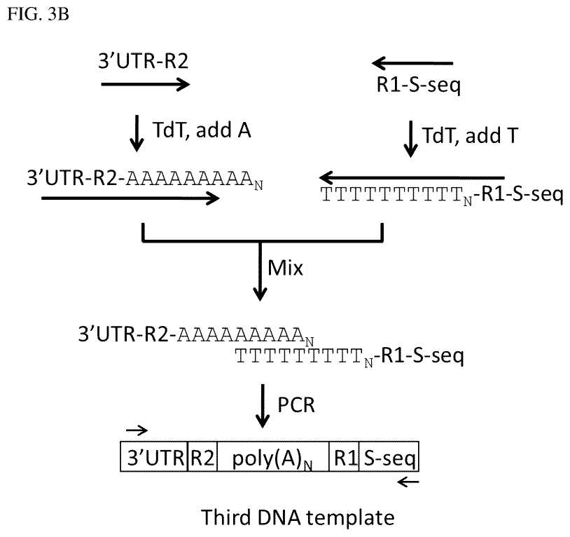

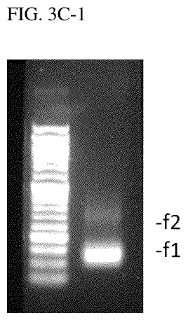

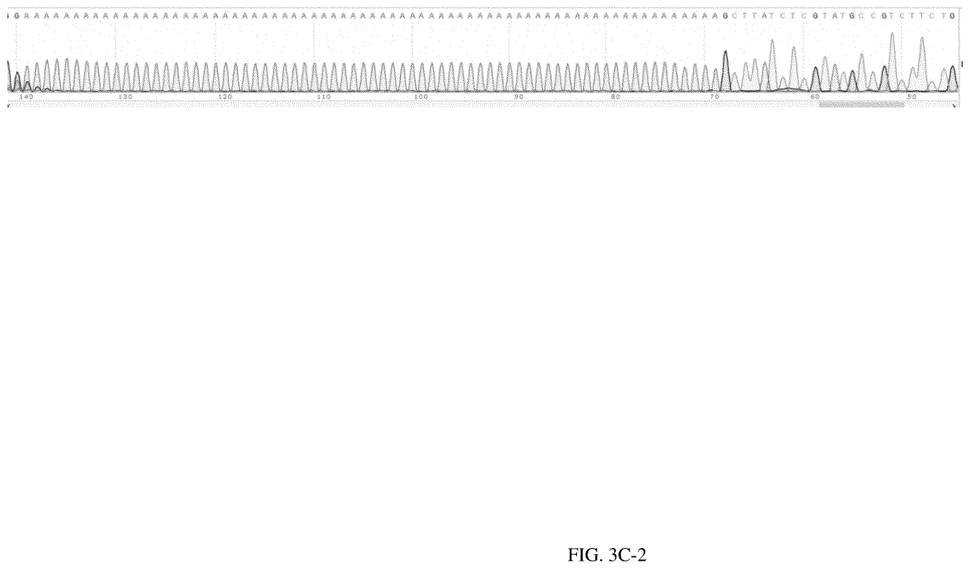

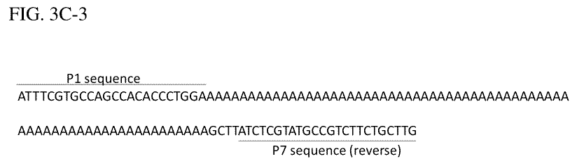

Method A: As shown in FIG. 3A, includes the following steps: Step 1: Use terminal deoxynucleotidyl transferase (TdT) to add poly(A) or poly(T) to the end of oligonucleotides, forming single-stranded Third DNA template sequences with different lengths of poly(A) or poly(T). Step 2: Mix the single-stranded Third DNA template sequences with poly(A) or poly(T) sequences and proceed to Step 3: Use PCR peripheral primers (i.e., primers used for the TdT reaction) to amplify the extended Third DNA template. Through PCR reaction, the Third DNA template with different lengths of poly(A) sequences can be amplified, obtaining amplified products of corresponding lengths, forming a Third DNA template with increased poly(A) sequence length. During the mixing process, poly(A) sequences of different lengths can combine to form an extended A sequence. The example shown in FIG. 3B introduces a second restriction enzyme cutting site, resulting in products with restriction enzyme cutting sites on both sides of the poly(A). This facilitates further extension of the poly(A) or the combination of the Second and Third DNA templates, which can be achieved through restriction enzyme cutting and ligation.

In Method A, the technique utilizes TdT in combination with primers, A and T nucleotides, allowing for the addition of poly(A) sequences of varying lengths to different region of the Third DNA template. Through mixing and PCR amplification, amplified products of the Third DNA template with different poly(A) lengths can be obtained.

This embodiment of Method A provides an effective method for increasing the length of the poly(A) in the Third DNA template. By controlling the length of the added poly(A) sequences and the amplification conditions of the PCR, the Third DNA template with poly(A) sequences of different lengths can be manipulated and utilized in various applications.

The results generated from Method A demonstrate that this method can amplify fragments with poly(A) sequences of varying lengths. Subsequently, these fragments of different lengths are directly cloned using a cloning vector.

### Example of Method A

TABLE 1
Primer Design for Method A

| | SEQ | |
|---|---|---|
| Primer | ID | |
| name | NO: | 5′-3′ |
| P1-A23 | 1 | ATTTCGTGCCAGCCACACCCTGGAAAAAAAA |
| | | AAAAAAAAAAAAAAAA |
| P7-T20 | 2 | CAAGCAGAAGACGGCATACGAGATAAGCTTT |
| | | TTTTTTTTTTTTTTTTTTT |
| P1 | 3 | ATTTCGTGCCAGCCACACCCTGGA |
| P7 | 4 | CAAGCAGAAGACGGCATACGAGAT |

Mix the components of the TdT reaction according to the following recipe: P1-A23 or P7-T20 primer 0.75 μl, 10× buffer 2.5 μl, 1 mM dATP or dTTP 0.5 μl, CoCl₂ 2.5 μl, TdT enzyme 2 μl, ddH₂O 16.75 μl, for a total volume of 25 μl. Add these components to the reaction tube and mix well. Incubate at 37° C. for 1.5 hours, followed by 10 minutes at 70° C. to inactivate the enzyme. Then, purify the reaction using the Gel/PCR purification mini kit (FAVOGEN, Taiwan, FAGCK 001-01). Take 1 μl of each purified product and mix them for PCR amplification (SMOBIO, Taiwan, TF1000). The reaction mixture includes: P1-A23 (TdT+A) 1 μl, P7-T20 (TdT+T) 1 μl, 10× HiFi™ Buffer 5 μl, MgSO₄ (25 mM) 2 μl, dNTPs (2 mM) 5 μl, P1 primer (10 μM) 1.5 μl, P7 primer (10 μM) 1.5 μl, SMO-HiFi™ DNA Polymerase 1 μl, and finally ddH₂O to a total volume of 50 μl. Mix well and perform PCR (Biometra, TProfessional basic). The PCR conditions are: initial denaturation at 94° C. for 2 minutes; followed by 32 cycles of 94° C. for 15 seconds, 56° C. for 15 seconds, and 68° C. for 30 seconds; with a final extension at 68° C. for 2 minutes.

After the PCR reaction is completed, confirm the PCR amplification products (f1 and f2) by electrophoresis on a 2% TAE agarose gel (FIG. 3C-1). Next, excise and purify the main bands f1 and f2 from the gel using the Gel/PCR purification mini kit (FAVOGEN, Taiwan, FAGCK 001-01). Clone the purified f1 and f2 fragments into the GetClone™ PCR Cloning Vector II, referred to as pGetII (SMOBIO, Taiwan, CV1100), and name the constructs pGetII-70A. Perform sequencing on the constructs, and the results (FIG. 3C-2) match the expected sequence (FIG. 3C-3).

Compared to the method of directly mixing and annealing primers P1-A23/P7-T20 followed by amplification using primers P1/P7, Method A first uses TdT enzyme. The enzyme adds more nucleotides (T or A sequences) to the ends of primers P1-A23/P7-T20 before PCR amplification, resulting in longer poly(A) fragments.

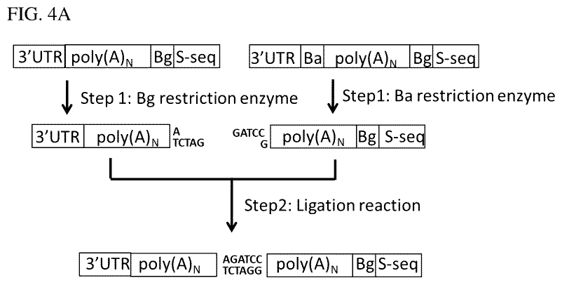

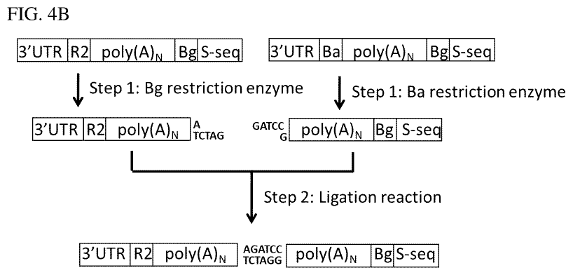

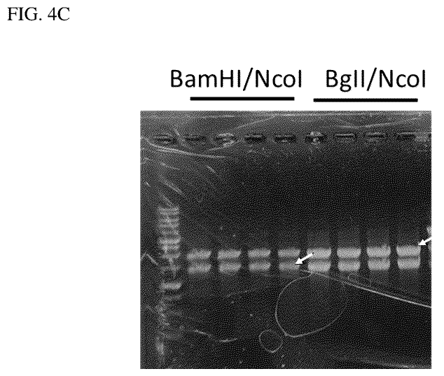

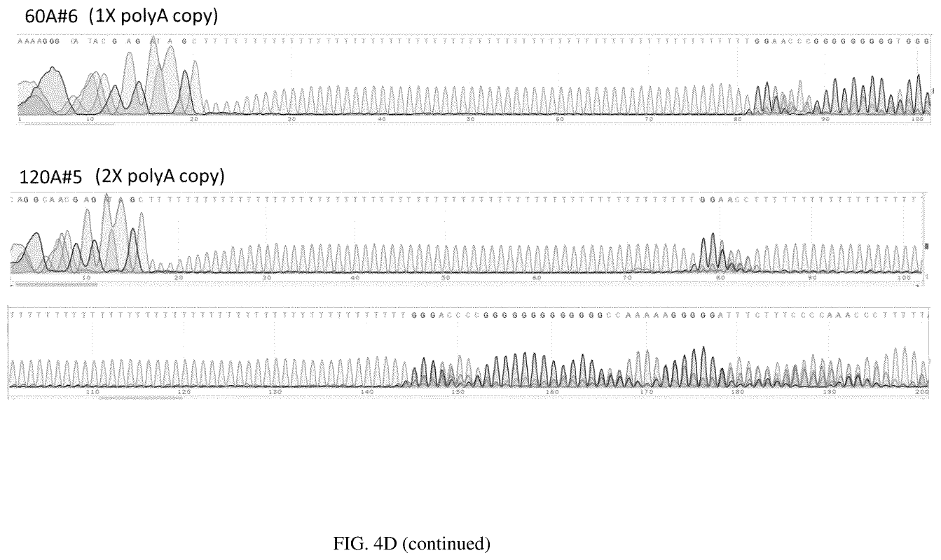

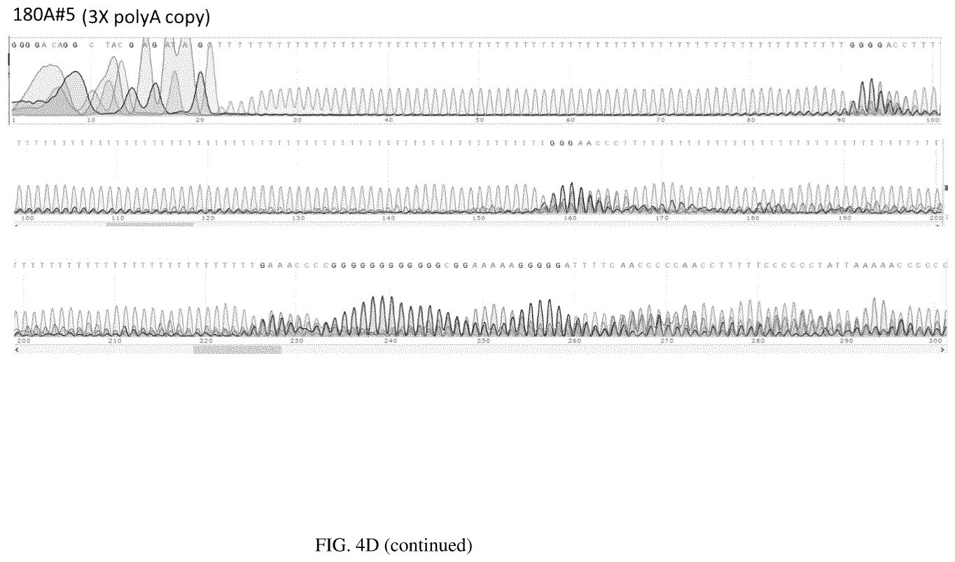

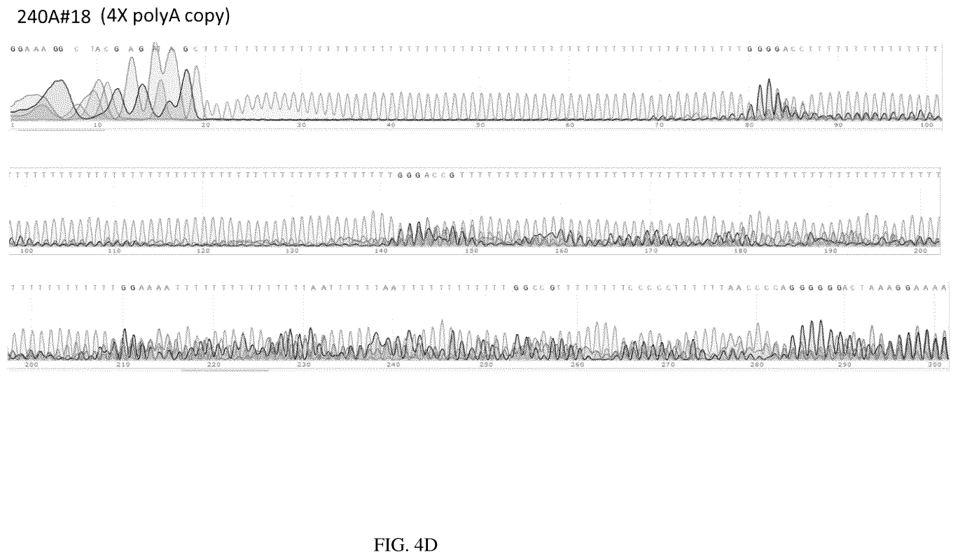

Method B provides a way to increase the length of poly(A) by designing different restriction enzyme cutting sites and repeating the processes of cutting and ligation. As shown in FIG. 4A, two Third DNA templates are designed. A first Third DNA template contains only the first restriction enzyme cutting site R1, which is BglII (Bg), while a second Third DNA template contains two restriction enzyme cutting sites, with the first restriction enzyme cutting site R1 being BglII (Bg) and the second restriction enzyme cutting site R2 being BamHI (Ba). Step 1: Use BglII to cut the first Third DNA template and BamHI to cut the second Third DNA template. The cut DNA fragments will expose complementary sticky ends. Step 2: Ligate the fragments with complementary sequences through a ligation reaction. After ligation, the sequence can no longer be recognized by BamHI and BglII, allowing the poly(A) length to be increased incrementally. By repeating this process, the poly(A) sequence in the Third DNA template can be gradually extended. FIG. 4B shows the first Third DNA template containing two restriction enzyme cutting sites, with the first restriction enzyme cutting site R1 being BglII, and the second restriction enzyme cutting site R2. The rest is the same as in FIG. 4A. The products of FIG. 4A and FIG. 4B, which are Third DNA templates with extended poly(A) sequences, can be further combined with the Second DNA template to obtain the First DNA template. This First DNA template can be used as an IVT transcription template.

In addition to the combination of BamHI and BglII, other combinations of restriction enzymes can also be used as long as the exposed sequences after cutting are complementary and can be ligated, and the ligated sequences can no longer be recognized by the original restriction enzymes. For example, combinations such as Spel and Nhel, AgeI and NgoMIV, NsiI and PstI, etc. These combinations provide various options to meet different experimental needs and the characteristics of the target DNA.

### Example of Method B

Table 2 is primer design for Method B.

TABLE 2
Primer design for Method B

| | SEQ | |
|---|---|---|
| Primer | ID | |
| name | NO: | 5′-3′ |
| P-BamHI- | 5 | ATTTCGTGCCAGCCACACCCTGGATCCA |
| polyA | | AAAAAAAA |
| M-Bglll- | 6 | CAAGCAGAAGACGGCATACGAGATAAG |
| polyA | | ATCTTTTTTTTT |
| M-S-Pvull | 7 | TGAGGGTATTCGTTACTCAAAGCC |
| P-GemA2 | 8 | CTGGTCATAGCTGTTTCCTGTCAGACCA |
| | | AG |

Modify the sequences on both sides of the poly A to include BamHI and BglII enzyme cutting sites by PCR method. This step is primarily used to add the BglII cutting site sequence and remove the multiple cloning site sequence connected to the BglII cutting site sequence on the vector. First, prepare the reaction mixture according to the following recipe: pGetII-70A 1 ng, 10× HiFi™ Buffer 5 μl, MgSO₄ (25 mM) 2 μl, dNTPs (2 mM each) 5 μl, P-GemA2 primer (10 μM) 1.5 μl, M-BglII-polyA primer (10 μM) 1.5 μl, SMO-HiFi™ DNA Polymerase 1 μl, and ddH₂O to a total volume of 50 μl. The PCR conditions: initial denaturation at 94° C. for 2 minutes; followed by 32 cycles of 94° C. for 15 seconds, 56° C. for 15 seconds, and 68° C. for 30 seconds; with a final extension at 68° C. for 2 minutes.

Next, purify the PCR product, and ligate the purified fragment, perform PCR again to add BamHI before polyA, and remove the multiple cloning site sequence of the vector. Prepare the PCR reaction mixture according to the following recipe: ligated DNA 1 ng, 10× HiFi™ Buffer 5 μl, MgSO₄ (25 mM) 2 μl, dNTPs (2 mM each) 5 μl, P-BamHI-polyA primer (10 μM) 1.5 μl, M-S-Pvull-primer (10 μM) 1.5 μl, SMO-HiFi™ DNA Polymerase 1 μl, and ddH₂O to a total volume of 50 μl. Add all components to the PCR reaction tube, mix well, and perform the PCR reaction. PCR reaction conditions: initial denaturation at 94° C. for 2 minutes; followed by 32 cycles of denaturation at 94° C. for 15 seconds, annealing at 56° C. for 15 seconds, and extension at 68° C. for 30 seconds; final extension at 68° C. for 2 minutes.

Next, purify the PCR product to obtain pGetII-60A-BB. Then perform double digestion with BamHI/NcoI and BglII/NcoI restriction enzymes, obtaining the results shown in FIG. 4C. Purify the gel products indicated by the arrows in FIG. 4C and perform ligation to further obtain DNA with 2×, 3×, and 4× polyA lengths. The sequencing results are shown in FIG. 4D. From the sequencing results, it can be observed that the original polyA template contained 70 A nucleotides, but using PCR to change the restriction enzyme cutting sites resulted in a shorter poly A length. In this example the poly A length is changed from 70 A nucleotides to 60 A nucleotides (see 60A #6 in FIG. 4D). After extending using BamHI and BglII cutting sites, the sequencing results showed that the polyA length increased as expected in multiples.

Method C provides a way to obtain a DNA template composed entirely of poly(A) sequences. Since poly(A) sequences themselves do not have suitable restriction enzyme cutting sites, a special method for ligation is required. Through Method C, it is possible to obtain a DNA template composed entirely of poly(A) sequences, with no non-poly(A) sequences at the junctions.

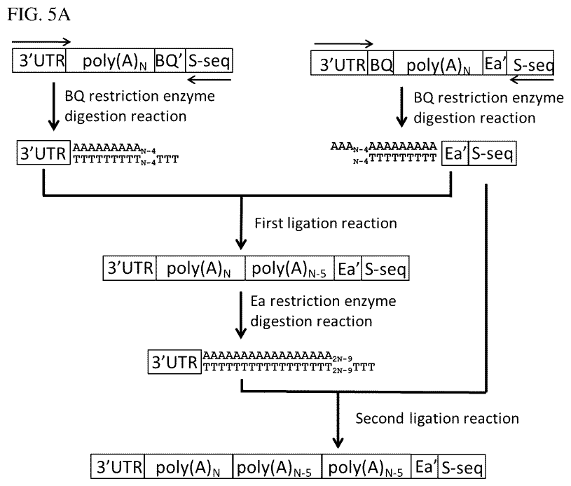

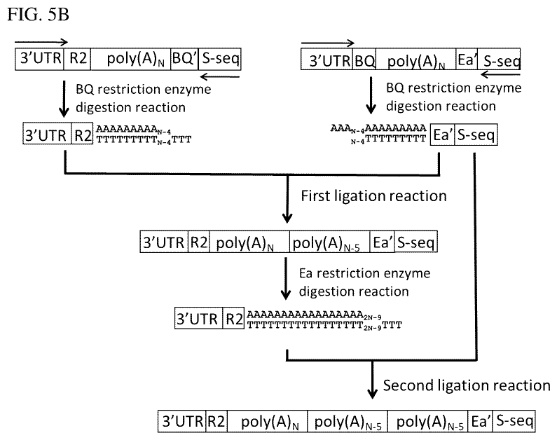

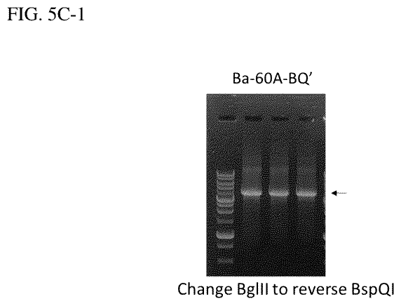

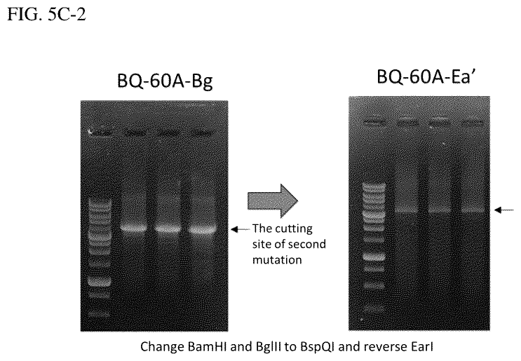

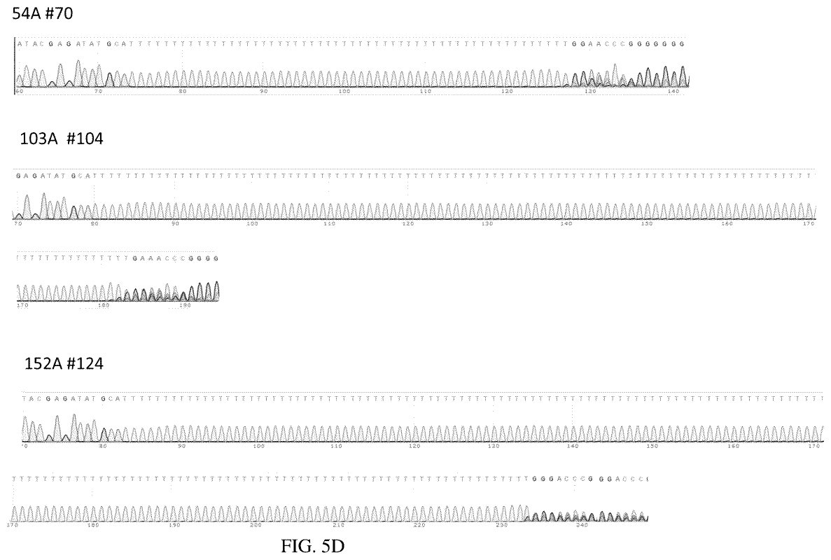

The specific method is as follows: Design a first Third DNA template with first restriction enzyme cutting site R1 as reverse BspQI (BQ′). Design a second Third DNA template with second restriction enzyme cutting site R2 as BspQI (BQ) and first restriction enzyme cutting site R1 as reverse EarI (Ea′). By separating the position of recognition sequences and cutting sites of BspQI (BQ) and EarI (Ea), sticky ends with protruding three T nucleotides or three A nucleotides can be generated. The protruding A nucleotides and T nucleotides will form complementary pairs, and the ligation will not introduce non-“A” sequences between the poly(A) sequences. The detailed process can be referred to in FIG. 5A. This process can be repeated. After the second ligation reaction, the resulting Third DNA template will contain a complete poly(A) sequence. This template can be ligated with the same 3′ untranslated region (3′UTR) on the Second DNA template or used in overlap PCR to obtain the complete First DNA template.

The experimental method of Method C is similar to Method B. However, since the poly A sequences ligated in Method B contain other non-polyA sequences (GGATCT), the recognition sequences and cutting sites of BspQI (BQ) and EarI (Ea) are offset to change the cutting sites.

Table 3 is primer design for Method C.

TABLE 3
Primer Design for Method C

| | SEQ | |
|---|---|---|
| | ID | |
| primer | NO: | 5′-3′ |
| P1-BspQIA-2 | 9 | ATTTCGTGCCAGCCACACCCTGGAGCTCT |
| | | TCAAAAAAAAA |
| P7-EarlA-2 | 10 | CAAGCAGAAGACGGCATACGAGATACTC |
| | | TTCTTTTTTTTT |
| M-S-Pvull | 7 | TGAGGGTATTCGTTACTCAAAGCC |
| P-GemA2 | 8 | CTGGTCATAGCTGTTTCCTGTCAGACCAA |
| | | G |
| P7-BspQIA-2 | 11 | CAAGCAGAAGACGGCATACGAGATGCTC |
| | | TTCTTTTTTTTT |
| M-8TNsi | 31 | CAAGCAGAAGACGGCATACGAGATATGC |
| | | ATTTTTTTT |

Detailed experimental procedures refer to the experimental content of Method B, with the key aspects related to Method C explained here.

(1) Replace the restriction enzyme cutting site BglII with reverse BspQI (BQ′): Using the vector pGetII-60A-BB obtained from Method B, use the primer pair P7-BspQIA-2/P-GemA2 and the PCR mutagenesis method to replace the BglII cutting site with reverse BspQI (BQ′). The electrophoresis results are shown in FIG. 5C-1. The resulting vector is named pGetII-60A-BaBQ′.

(2) Replace the BamHI and BglII cutting sites with BspQI and reverse EarI: Using the vector pGetII-60A-BB obtained from Method B, first use the primer pair P1-BspQIA-2/M-S-PvuII and PCR to replace the BamHI cutting site with BspQI. Then use another primer pair P7-EarIA-2/P-GemA2 to replace the BglII cutting site with reverse EarI (Ea′). The electrophoresis results are shown in FIG. 5C-2. The resulting vector is named pGetII-60A-BsEa′. (3) Cut pGetII-60A-BaBQ′ with BspQI and NcoI restriction enzymes, and cut pGetII-60A-BsEa′ with EarI and NcoI restriction enzymes. Next, purify the cutted fragment products and perform ligation. Repeat the above steps to sequentially obtain fragments containing 2×, 3×, and 4× polyA. The difference in the fragments obtained by this method compared to those from Method B is that with each additional poly A fragment, 5 A nucleotides are reduced.

Mutate the restriction enzyme recognition sequences (BspQI or EarI) on the 1×, 2×, and 3× polyA fragments to NsiI restriction enzyme recognition sequences using the primer pair (M-8TNsi and P-GemA2) through PCR. This is because NsiI is easier to use for restriction enzyme digestion compared to BspQI or EarI. The sequencing results are shown in FIG. 5D, where the 1× poly A has 54 A nucleotides, the 2× polyA has 103 A nucleotides, and the 3× poly A has 152 A nucleotides. However, the 1× polyA changed from 60 A nucleotides to 54 A nucleotides, possibly due to deletions occurring during the PCR process or within the bacterial host.

Through Method C, a DNA template containing a complete poly(A) sequence can be obtained, with no non-poly(A) sequences at the junctions. This is particularly useful for experiments or applications that require pure poly(A) sequences as the target. This method can also be applied to the design of pure poly(G), poly(C), poly(T), and high-length sequences.

Method D provides a way to obtain a DNA template composed entirely of poly(A) sequences, with no non-poly(A) sequences at the junctions. The initial poly(A) sequence can directly start from the primer and gradually extend from shorter poly(A) sequences.

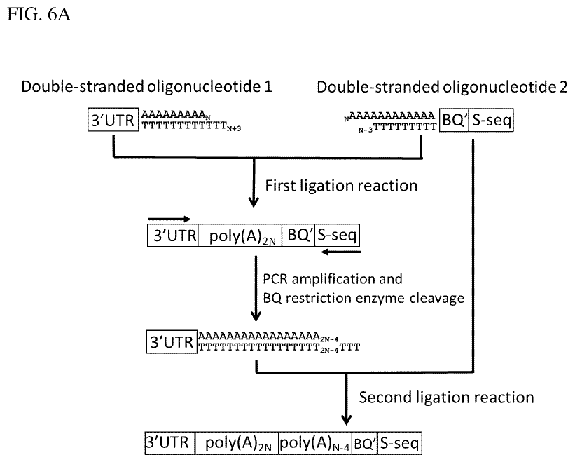

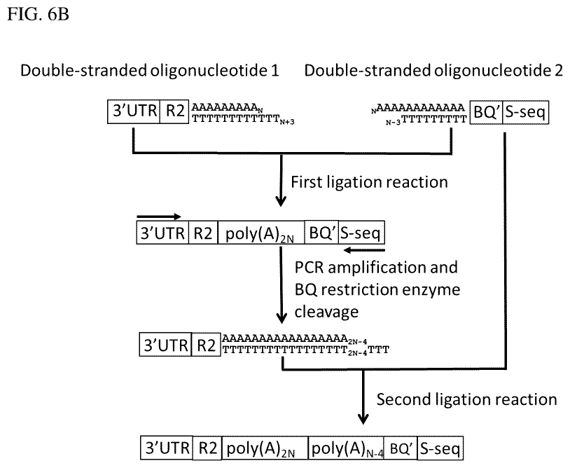

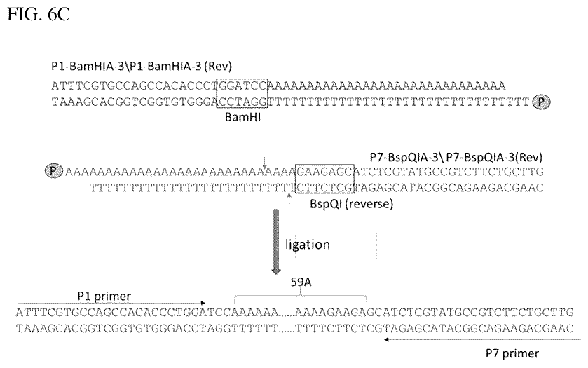

The specific method is as follows: Design the first double-stranded oligonucleotide pairing sequence, composed of two complementary longer oligonucleotides. The sequence includes a portion of the 3′ untranslated region and a fixed-length but unlimited-length A/T pairing, with a protruding three T nucleotides. Provide the second double-stranded oligonucleotide pairing sequence, including a fixed-length A/T pairing, a BspQI restriction enzyme cutting site, and a specific sequence (S-seq), with a protruding three A nucleotides at the A/T pairing position. This short nucleotide sequence can also be composed of two artificially synthesized longer oligonucleotides. Phosphorylate the 5′ ends of the primers with protruding three T nucleotides and three A nucleotides in these two double-stranded oligonucleotides pairing sequences. Mix the two short nucleotide sequences, and due to the complementarity of AAA and TTT, ligation can occur, resulting in an extended polyadenylate. The extended polyadenylate sequence can be cut with BspQI to obtain a product with a protruding three T nucleotides. This product can be further ligated with the second double-stranded oligonucleotide pairing sequence with a protruding three A nucleotides, thereby extending the poly(A) length further. Repeat the above steps to continue extending the polyadenylate length. This method can also design a second restriction enzyme cutting site in the first short nucleotide sequence, as shown in FIG. 6B. The resulting Third DNA template can be combined with the Second DNA template through various methods, such as ligation, restriction enzyme cutting, and overlap PCR, as previously described.

Through Method D, a DNA template containing a complete poly(A) sequence can be obtained, with no non-poly(A) sequences at the junctions This method yields results similar to those of Method C and can also be applied to the design of pure poly(G), poly(C), poly(T), and high-length sequences.

The experimental method of Method D is similar to Methods B and C. The designed primer pairs are shown in Table 4, with lengths below 60 mer, which is the maximum length for conventional synthesis.

TABLE 4
Primer design of Method D

| | SEQ | |
|---|---|---|
| | ID | |
| primer | NO: | 5′-3′ |
| P1- | 12 | ATTTCGTGCCAGCCACACCCTGGATCCAA |
| BamHIA-3 | | AAAAAAAAAAAAAAAAAAAAAAAAAAAA |
| P1- | 13 | TTTTTTTTTTTTTTTTTTTTTTTTTTTTTTTTT |
| BamHIA- | | GGATCCAGGGTGTGGCTGGCACGAAAT |
| 3(Rev) | | |
| P7-BspQIA- | 14 | AAAAAAAAAAAAAAAAAAAAAAAAAAAA |
| 3 | | AGAAGAGCATCTCGTATGCCGTCTTCTGCT |
| | | TG |
| P7-BspQIA- | 15 | CAAGCAGAAGACGGCATACGAGATGCTCT |
| 3(Rev) | | TCTTTTTTTTTTTTTTTTTTTTTTTTTT |
| P1 | 3 | ATTTCGTGCCAGCCACACCCTGGA |
| P7 | 4 | CAAGCAGAAGACGGCATACGAGAT |

The first double-stranded oligonucleotide pairing sequence can be composed of the primers P1-BamHIA-3/P1-BamHIA-3 (Rev), and the second double-stranded oligonucleotide pairing sequence can be composed of the primers P7-BspQIA-3/P7-BspQIA-3 (Rev).

The experimental procedure for pairing the double-stranded sequences is as follows:

(1) Primer Phosphorylation: Phosphorylate individually the primers P1-BamHIA-3 (Rev) and P7-BspQIA-3 using T4 PNK (NEB, M0201S). Prepare the reaction mixture according to the following recipe: P1-BamHIA-3 (Rev) primer or P7-BspQIA-3 primer (10 μM) 10 μl, T4 PNK Reaction Buffer (10×) 5 μl, ATP (10 mM) 5 μl, T4 PNK (10 units/μl) 1 μl, Nuclease-free Water 29 μl, for a total reaction volume of 50 μl. Mix well and incubate at 37° C. for 30 minutes, then inactivate the enzyme by incubating at 65° C. for 20 minutes.

(2) Forming double-stranded oligonucleotide pairs: Prepare the following reaction mixtures, including: First set of primers: P1-BamH1A-3 (10 μM) 10 μl and phosphorylated P1-BamH1A-3 (Rev) (2 μM) 50 μl. Second set of primers: phosphorylated P7-BspQ1A-3 (2 μM) 50 μl and P7-Ear1A-3 (Rev) (10 μM) 10 μl.

(3) Using a thermal cycler (Biometra, TProfessional basic), set the temperature reactions as follows: first denature at 95° C. for 2 minutes, then decrease the temperature by approximately 1 degree per minute to allow the primers to anneal automatically. The temperature settings for this embodiment are 95° C. for 2 minutes; 90° C. for 5 minutes; 85° C. for 5 minutes; 80° C. for 5 minutes; 75° C. for 5 minutes; 70° C. for 5 minutes; 65° C. for 5 minutes; 60° C. for 5 minutes; 55° C. for 5 minutes; and 50° C. for 5 minutes. After the reaction, place on ice to obtain the first double-stranded oligonucleotide pairing sequence and the second double-stranded oligonucleotide pairing sequence. The double-stranded DNA structures are shown in FIG. 6C.

Ligate the first double-stranded oligonucleotide pairing sequence with the second double-stranded oligonucleotide pairing sequence using Ligation high (TOYOBO, Japan, LGK-101). Prepare the following reaction mixture: P1-BamH1-3/P1-BamH1A-3 (Rev) dsDNA 5 μl, P7-BspQ1A-3/P7-BspQ1A-3 (Rev) dsDNA 5 μl, and Ligation high 5 μl, for a total reaction volume of 15 μl. The reaction conditions are: set the reaction temperature to 25° C. for 30 minutes, then place the reaction mixture at 4° C. At this point, the two double-stranded oligonucleotides are paired, and their final sequence is shown in FIG. 6C. Next, amplify the paired oligonucleotides using the outer primers (P1/P7). Prepare the PCR reaction mixture according to the following recipe: ligation product 1 ng, 10× HiFi™ Buffer 5 μl, MgSO₄ (25 mM) 2 μl, dNTPs (2 mM each) 5 μl, P1 primer (10 μM) 1.5 μl, P7 primer (10 μM) 1.5 μl, SMO-HiFi™ DNA Polymerase 1 μl, and ddH₂O to a total volume of 50 μl. The PCR reaction conditions are initial denaturation at 94° C. for 2 minutes; followed by 32 cycles of denaturation at 94° C. for 15 seconds, annealing at 56° C. for 15 seconds, and extension at 68° C. for 30 seconds; with a final extension at 68° C. for 1 minute.

The PCR product size is 117 bp (containing 59 A nucleotides). Purify the PCR product and increase the polyA sequence using the method described in Method C. As shown in FIG. 6A and FIG. 6B, digest the amplified PCR product with BspQI restriction enzyme. Purify the digested product (84 bp) and further ligate it with the second double-stranded oligonucleotide pairing sequence P7-BspQIA-3/P7-BspQIA-3 (Rev) dsDNA. Amplify the ligated product using PCR with the outer primers (P1/P7). The amplified product size is 142 bp (containing 84 A nucleotides). Each experimental cycle increases the poly A length by 25 A nucleotides.

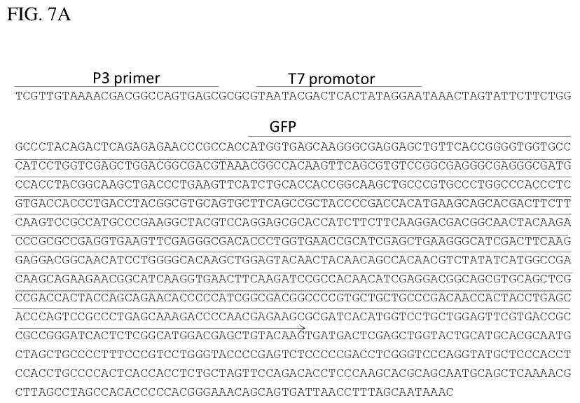

In addition to the methods mentioned above, those skilled in the art can perform the ligation reaction of two fragments using restriction enzyme ligation techniques or overlap PCR methods as disclosed in current molecular cloning technologies. The present invention provides some experimental results, where poly A fragments of different sizes are combined using overlap PCR with a DNA fragment containing a T7 promoter, 5′ UTR, the complete green fluorescent protein gene open reading frame (GFP, ORF), and 3′ UTR, as shown in FIG. 7A. After combining this sequence with DNA fragments of different polyA lengths, perform PCR using the primer pair P3/P7 (Table 5) to amplify fragments containing GFP and poly A of varying lengths, as shown in FIG. 7B.

TABLE 5

| primer | SEQ ID NO: | 5′-3′ |
|---|---|---|
| P7 | 4 | CAAGCAGAAGACGGCATACGAGAT |
| P3 | 16 | ACGTTGTAAAACGACGGCCAGTGA |

This fragment can further be constructed into a DNA template for RNA production, capable of expressing green fluorescent protein in mammalian cell lines. Prepare the PCR reaction mixture according to the following recipe: GFP 1 ng, polyA fragment 1 ng, 10× HiFi™ Buffer 5 μl, MgSO₄ (25 mM) 2 μl, dNTPs (2 mM each) 5 μl, P3 primer (10 μM) 1.5 μl, P7 primer (10 μM) 1.5 μl, SMO-HiFi™ DNA Polymerase 1 μl, and ddH₂O to a total volume of 50 μl. Add all components to the PCR reaction tube, mix well, and perform the PCR reaction. The PCR reaction conditions: initial denaturation at 94° C. for 2 minutes; followed by 32 cycles of denaturation at 94° C. for 15 seconds, annealing at 56° C. for 15 seconds, and extension at 68° C. for 3 minutes; with a final extension at 68° C. for 3 minutes.

The fragments amplified using the above method can be used for in vitro transcription (IVT) as needed. To preserve the DNA, the fragments can be cloned into a plasmid for storage. As shown in FIG. 7C, the GFP fragments with different poly A lengths currently assembled in this case have a theoretical polyA length of up to 397 A bases.

Furthermore, the number of polyadenylate sequences in the Third DNA template can extend to over 1000 nucleotides (i.e., N=1000). Using the methods disclosed in this case, such as Method A, B, C, or D, the length can be adjusted according to the actual length of the mRNA product. Therefore, the DNA transcription template production method disclosed by the present invention is a simple and flexible method for producing DNA templates.

In this embodiment, the GFP gene with different lengths of poly A, including pGet-GFP-54A-NsiI, pGet-GFP-103A-NsiI, and pGet-GFP-152A-NsiI, each containing 54, 103, and 152 A bases of polyA respectively, are cut with NsiI and then used for in vitro transcription (IVT) with the IVT reagent kit (Thermo, #K0441). Prepare the following reaction mixture: 5× TranscriptAid Reaction buffer 4 μl, ATP/CTP/UTP mix (25 mM each) 4 μl, Template DNA 1 μg, TranscriptAid Enzyme Mix 2 μl, and DEPC-treated water to a final volume of 20 μl.

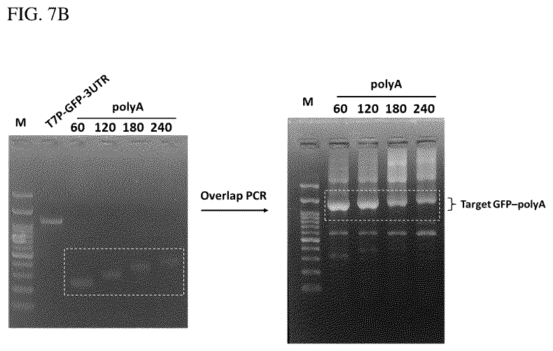

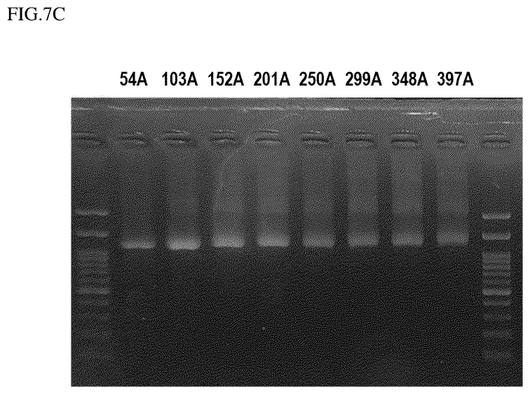

After the IVT reaction, perform the capping reaction on the purified RNA using Vaccinia Capping Enzymes (NEB, M2080S) and Vaccinia 2′-O-Methyltransferase (2′-OME) (NEB, M0366S). Step 1: Denature the RNA. Prepare the reaction mixture as follows: Purified RNA (uncapped) 10 μg and Nuclease-Free Water to a total reaction volume of 13 μl. Incubate at 65° C. for 5 minutes, then place on ice. Step 2: Perform the capping reaction. Prepare the reaction mixture as follows: Denatured RNA 13 μl, 10× Capping Buffer 2 μl, GTP (10 mM) 1 μl, SAM (4 mM, diluted from 32 mM stock) 1 μl, Vaccinia Capping Enzyme 1 μl, 2′-O-Methyltransferase 1 μl, RNase Inhibitor 1 μl, for a total volume of 20 μl. Incubate at 37° C. for 1 hour. Analyze the samples by 1% agarose gel electrophoresis, as shown in FIG. 8.

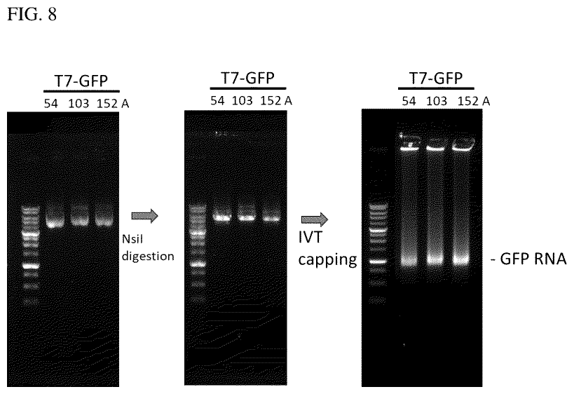

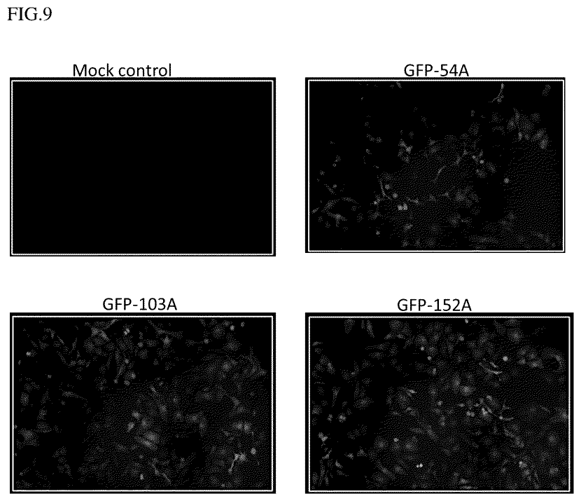

Cell transfection experiment: In vitro synthesized RNA with different lengths, specifically GFP mRNA with 54 A bases, 103 A bases, and 152 A bases, were transfected into HeLa cells using 1 μg of RNA and Lipofectamine transfection reagent (Thermo, 11668019). The fluorescence results were observed using a fluorescence microscope (Motic, model AE31E) (FIG. 9) and a flow cytometer (BD, FACSLyric). The fluorescence intensity quantification results are shown in Table 6.

TABLE 6
Quantification results of sample fluorescence

| Sample | Count | Median fluorescent |
|---|---|---|
| Mock control | 7201 | 44674 |
| GFP-54A | 13206 | 5823412 |
| GFP-103A | 13502 | 8792819 |
| GFP-152A | 13755 | 10440255 |

What is claimed is:

1. A method for producing a DNA template, wherein the DNA template is used for producing mRNA containing a polyadenylate tail, the DNA template comprising:
(a) Providing a First DNA template, wherein the First DNA template comprises, in sequence from the 5′ end to the 3′ end, a forward primer sequence, a promoter, a target DNA, wherein the target DNA further comprises a 5′ untranslated region and a 3′ untranslated region, a polyadenylate, a first restriction enzyme cutting site, and a specific sequence, wherein the specific sequence is a reverse primer complementary sequence;
(b) Using the First DNA template as template, adding a forward primer and a reverse primer to perform PCR amplification of the First DNA template; and
(c) Performing a cleavage on amplified the First DNA template using the first restriction enzyme, wherein the cleavage removes the specific sequence of the First DNA template to obtain the DNA template.

2. The method of claim 1, wherein the First DNA template further comprises a second restriction enzyme (R2) cutting site between the 3′ untranslated region and the polyadenylate.

3. The method of claim 1, wherein a method for producing the First DNA template is obtained through ligation reaction or overlap PCR using a Second DNA template and a Third DNA template; wherein the Second DNA template comprises, in sequence from the 5′ end to the 3′ end, a forward primer sequence, a promoter, a target DNA, wherein the target DNA further comprises a 5′ untranslated region and a 3′ untranslated region; wherein the Third DNA template comprises, in sequence from the 5′ end to the 3′ end, the same or a continuous 3′ untranslated region sequence as the Second DNA template, a polyadenylate, a first restriction enzyme cutting site, and a specific sequence, wherein the specific sequence is a reverse primer complementary sequence.

4. The method of claim 1, wherein the length of the polyadenylate is greater than 200 nucleotides, greater than 300 nucleotides, greater than 400 nucleotides, greater than 500 nucleotides, greater than 600 nucleotides, or greater than 1000 nucleotides.

5. The method of claim 2, wherein the overlap PCR is performed through overlapping region of the 3′ untranslated region present on both the Second DNA template and the Third DNA template to carry out the PCR amplification of the complete DNA fragment.

6. The method of claim 2, wherein the ligation reaction is performed by the blunt-end ligation or the use of the same second restriction enzyme, through the contiguous sequence of the 3′ untranslated region present on both the Second DNA template and the Third DNA template, to achieve the ligation of the Second DNA template and the Third DNA template,
wherein the restriction enzyme cutting site can be an inherent restriction enzyme cutting site of the 3′ untranslated region or an inserted restriction enzyme cutting site designed for cleavage and ligation.

7. A method for increasing length of the polyadenylate in the Third DNA template in the method of claim 2, comprising the steps of:
(a) Using terminal deoxynucleotidyl transferase to add nucleotides to the primers as the end of the Third DNA template;
(b) Adding polyadenylate or polythymidylate sequences of different lengths to the end of the primers, generating a single-stranded third DNA sequences with one or more polyadenylate or polythymidylate sequences of different lengths;
(c) Mixing the single-stranded third DNA sequences with one or more polyadenylate or polythymidylate sequences to form the Third DNA template with an increased length of adenylate sequence, wherein the Third DNA template with an increased length of adenylate sequence comprises polyadenylate sequences of different lengths; and
(d) Amplifying the Third DNA template with an increased length of adenylate sequence by PCR to obtain the Third DNA template with an increased length of adenylate sequence.
(e) This method is also applicable to designs containing a second restriction enzyme (R2) cutting site, where the cutting site is between the 3′ untranslated region and a polyadenylate.

8. A method for increasing the length of the polyadenylate in the Third DNA template in the method of claim 2, comprising the steps of:
(a) Providing a first Third DNA template, adding a first restriction enzyme to perform a cleavage, generating the first Third DNA template with the first restriction enzyme cleavage, wherein the first Third DNA template with the first restriction enzyme cleavage exposes a sequence cleaved by the first restriction enzyme;
(b) Providing a second Third DNA template, adding a second restriction enzyme to perform a cleavage, generating the second Third DNA template with the second restriction enzyme cleavage, wherein the second Third DNA template with a second restriction enzyme cleavage exposes a sequence cleaved by the second restriction enzyme, wherein the sequences cleaved by the first and the second restriction enzymes are complementary sequences; and
(c) Performing a ligation reaction between the first Third DNA template with the first restriction enzyme cleavage and the second Third DNA template with the second restriction enzyme cleavage, obtaining the Third DNA template with increased polyadenylate length.
(d) This method is also applicable to designs containing a second restriction enzyme (R2) cutting site, where the cutting site is between the 3′ untranslated region and a polyadenylate.

9. A method for increasing the length of the polyadenylate in the Third DNA template in the method of claim 2, comprising the steps of:
(a) Providing a first Third DNA template, wherein the first restriction enzyme cutting site is reverse BspQI sequence; providing a second Third DNA template, wherein the first restriction enzyme cutting site is reverse Earl sequence, and the second restriction enzyme cutting site is BspQI sequence;
(b) Cleaving the first Third DNA template with BspQI, generating the first Third DNA template with BspQI cleavage, wherein the cleaved sequence has protruding TTT sequence;
(c) Cleaving the second Third DNA template with BspQI, generating the second Third DNA template with BspQI cleavage, wherein the cleaved sequence has protruding AAA sequence;
(d) Ligation of the first Third DNA template with BspQI cleavage and the second Third DNA template with BspQI cleavage, obtaining a third Third DNA template;
(e) Cleaving the third Third DNA template with Earl, generating the third Third DNA template with Earl cleavage, wherein the cleaved sequence has protruding TTT sequence; and
(f) Performing a ligation reaction between the third Third DNA template with Earl cleavage and the second Third DNA template with BspQI cleavage, obtaining the Third DNA template with increased polyadenylate length.
(g) This method is also applicable to designs containing a second restriction enzyme (R2) cutting site, where the cutting site is between the 3′ untranslated region and a polyadenylate.

10. A method for increasing the length of the polyadenylate in the Third DNA template in the method of claim 2, comprising the steps of:
(a) Providing a first double-stranded oligonucleotide pairing sequence, comprising a 3′ untranslated region sequence and a fixed length of A/T pairing, and protruding three T nucleotides; providing a second double-stranded oligonucleotide pairing sequence, comprising a fixed length of A/T pairing, a BspQI restriction enzyme cutting site, and a specific sequence (S-seq), and the position of the A/T pairing is designed to protrude three A nucleotides;
(b) Phosphorylating the 5′ end of the primer with the protruding three T nucleotides in the first double-stranded oligonucleotide pairing sequence;
(c) Phosphorylating the 5′ end of the primer with the protruding three A nucleotides in the second double-stranded oligonucleotide pairing sequence;
(d) Ligation of the first double-stranded oligonucleotide pairing sequence and the second double-stranded oligonucleotide pairing sequence to obtain a third Third DNA template;
(e) Amplifying the third Third DNA template using the outer primer pair by PCR and cleaving with BspQI, generating the Third DNA template with a BspQI cleavage, wherein the cleaved sequence has a protruding TTT sequence; and
(f) Performing a ligation reaction between the third Third DNA template with a BspQI cleavage and the second double-stranded oligonucleotide pairing sequence with a protruding three A nucleotides, obtaining the Third DNA template with an increased polyadenylate length.
(g) This method is also applicable to designs containing a second restriction enzyme (R2) cutting site, where the cutting site is between the 3′ untranslated region and a polyadenylate.


SEQUENCE LISTING (31 sequences)

SEQ ID NO: 1 - DNA, 47 bp, synthetic construct
  source 1..47: mol_type = other DNA
atttcgtgccagccacaccctggaaaaaaaaaaaaaaaaaaaaaaaa

SEQ ID NO: 2 - DNA, 50 bp, synthetic construct
  source 1..50: mol_type = other DNA
caagcagaagacggcatacgagataagctttttttttttttttttttttt

SEQ ID NO: 3 - DNA, 24 bp, synthetic construct
  source 1..24: mol_type = other DNA
atttcgtgccagccacaccctgga

SEQ ID NO: 4 - DNA, 24 bp, synthetic construct
  source 1..24: mol_type = other DNA
caagcagaagacggcatacgagat

SEQ ID NO: 5 - DNA, 36 bp, synthetic construct
  source 1..36: mol_type = other DNA
atttcgtgccagccacaccctggatccaaaaaaaaa

SEQ ID NO: 6 - DNA, 39 bp, synthetic construct
  source 1..39: mol_type = other DNA
caagcagaagacggcatacgagataagatcttttttttt

SEQ ID NO: 7 - DNA, 24 bp, synthetic construct
  source 1..24: mol_type = other DNA
tgagggtattcgttactcaaagcc

SEQ ID NO: 8 - DNA, 30 bp, synthetic construct
  source 1..30: mol_type = other DNA
ctggtcatagctgtttcctgtcagaccaag

SEQ ID NO: 9 - DNA, 40 bp, synthetic construct
  source 1..40: mol_type = other DNA
atttcgtgccagccacaccctggagctcttcaaaaaaaaa

SEQ ID NO: 10 - DNA, 40 bp, synthetic construct
  source 1..40: mol_type = other DNA
caagcagaagacggcatacgagatactcttcttttttttt

SEQ ID NO: 11 - DNA, 40 bp, synthetic construct
  source 1..40: mol_type = other DNA
caagcagaagacggcatacgagatgctcttcttttttttt

SEQ ID NO: 12 - DNA, 57 bp, synthetic construct
  source 1..57: mol_type = other DNA
atttcgtgccagccacaccctggatccaaaaaaaaaaaaaaaaaaaaaaaaaaaaaa

SEQ ID NO: 13 - DNA, 60 bp, synthetic construct
  source 1..60: mol_type = other DNA
tttttttttttttttttttttttttttttttttggatccagggtgtggctggcacgaaat

SEQ ID NO: 14 - DNA, 60 bp, synthetic construct
  source 1..60: mol_type = other DNA
aaaaaaaaaaaaaaaaaaaaaaaaaaaaagaagagcatctcgtatgccgtcttctgcttg

SEQ ID NO: 15 - DNA, 57 bp, synthetic construct
  source 1..57: mol_type = other DNA
caagcagaagacggcatacgagatgctcttctttttttttttttttttttttttttt

SEQ ID NO: 16 - DNA, 24 bp, synthetic construct
  source 1..24: mol_type = other DNA
acgttgtaaaacgacggccagtga

SEQ ID NO: 17 - DNA, 96 bp, synthetic construct
  source 1..96: mol_type = other DNA
gaaaaaaaaaaaaaaaaaaaaaaaaaaaaaaaaaaaaaaaaaaaaaaaaaaaaaaaaaaaaaaaaaaaaaaagcttatctcgtatgccgtcttctg

SEQ ID NO: 18 - DNA, 119 bp, synthetic construct
  source 1..119: mol_type = other DNA
atttcgtgccagccacaccctggaaaaaaaaaaaaaaaaaaaaaaaaaaaaaaaaaaaaaaaaaaaaaaaaaaaaaaaaaaaaaaaaaaaagcttatctcgtatgccgtcttctgcttg

SEQ ID NO: 19 - DNA, 101 bp, synthetic construct
  source 1..101: mol_type = other DNA
aaaagggcatacgagatagctttttttttttttttttttttttttttttttttttttttttttttttttttttttttttttggaacccgggggggggtggg

SEQ ID NO: 20 - DNA, 200 bp, synthetic construct
  source 1..200: mol_type = other DNA
caggcaacgagatagctttttttttttttttttttttttttttttttttttttttttttttttttttttttttttttggaacctttttttttttttttttttttttttttttttttttttttttttttttttttttttttttttgggaccccgggggggggggggccaaaaagggggatttctttccccaaacccttttt

SEQ ID NO: 21 - DNA, 301 bp, synthetic construct
  source 1..301: mol_type = other DNA
ggggacaggctacgagatagctttttttttttttttttttttttttttttttttttttttttttttttttttttttttttttttttttttggggaccttttttttttttttttttttttttttttttttttttttttttttttttttttttttttttgggaacccttttttttttttttttttttttttttttttttttttttttttttttttttttttttttttgaaaccccggggggggggggcggaaaaagggggattttcaacccccaacctttttcccccctattaaaaacccccc

SEQ ID NO: 22 - DNA, 301 bp, synthetic construct
  source 1..301: mol_type = other DNA
ggaaaggctacgagatagcttttttttttttttttttttttttttttttttttttttttttttttttttttttttttttggggacctttttttttttttttttttttttttttttttttttttttttttttttttttttttgggaccgtttttttttttttttttttttttttttttttttttttttttttttttttttttttttttttggaaaatttttttttttttttaattttttaattttttttttttggccgttttttttcccccttttttaaccccaggggggactaaaggaaaa

SEQ ID NO: 23 - DNA, 82 bp, synthetic construct
  source 1..82: mol_type = other DNA
atacgagatatgcattttttttttttttttttttttttttttttttttttttttttttttttttttttggaacccggggggg

SEQ ID NO: 24 - DNA, 124 bp, synthetic construct
  source 1..124: mol_type = other DNA
gagatatgcatttttttttttttttttttttttttttttttttttttttttttttttttttttttttttttttttttttttttttttttttttttttttttttttttttttttgaaacccgggg

SEQ ID NO: 25 - DNA, 179 bp, synthetic construct
  source 1..179: mol_type = other DNA
tacgagatatgcattttttttttttttttttttttttttttttttttttttttttttttttttttttttttttttttttttttttttttttttttttttttttttttttttttttttttttttttttttttttttttttttttttttttttttttttttttttttgggacccgggaccc

SEQ ID NO: 26 - DNA, 33 bp, synthetic construct
  source 1..33: mol_type = other DNA
atttcgtgccagccacaccctggatccaaaaaa

SEQ ID NO: 27 - DNA, 35 bp, synthetic construct
  source 1..35: mol_type = other DNA
aaaagaagagcatctcgtatgccgtcttctgcttg

SEQ ID NO: 28 - DNA, 33 bp, synthetic construct
  source 1..33: mol_type = other DNA
taaagcacggtcggtgtgggacctaggtttttt

SEQ ID NO: 29 - DNA, 35 bp, synthetic construct
  source 1..35: mol_type = other DNA
ttttcttctcgtagagcatacggcagaagacgaac

SEQ ID NO: 30 - DNA, 1050 bp, synthetic construct
  source 1..1050: mol_type = other DNA
tcgttgtaaaacgacggccagtgagcgcgcgtaatacgactcactataggaataaactagtattcttctggtccctacagactcagagagaacccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaac

SEQ ID NO: 31 - DNA, 37 bp, synthetic construct
  source 1..37: mol_type = other DNA
caagcagaagacggcatacgagatatgcatttttttt